(19) United States
(12) Patent Application Publication
(10) Pub. No.: US 2009/0228032 A1
(43) Pub. Date: Sep. 10, 2009

(54) ULTRASONIC SCALPEL

(75) Inventor: Eilaz BABAEV, Minnetonka, MN (US)

Correspondence Address: Bacoustics, LLC, 5929 BAKER ROAD, SUITE 470, MINNETONKA, MN, 55345, US

(73) Assignee: Bacoustics, LLC, Minnetonka, MN (US)

(21) Appl. No.: 12/043,173

(22) Filed: Mar. 6, 2008

(51) Int. Cl. A61B 17/3211

(52) U.S. Cl. 606/169

(57) ABSTRACT

An ultrasound scalpel that may be used for surgical operations is disclosed in this invention. The ultrasound scalpel comprises an ultrasound generator, housing, transducer horn, retaining member and scalpel blade. The device is able to utilize sharp stainless steel surgical blades amenable to being produced as disposable single-use replacement blades. Ultrasonic energy transmitted from transducer horn to surgical blade causes bending waves to be created due to the off-center attachment and/or placement of the surgical blade. This device may be utilized for cutting through skin and/other soft tissues during surgical operations, thereby enhancing cutting efficacy, decreasing and/or eliminating necrosis formation.

### BACKGROUND OF THE INVENTION

This present invention relates generally to a surgical instrument that can use a disposable blade, and more particularly, to an ultrasonic scalpel having improved cutting abilities during surgical operations involving cutting through skin and/or soft tissue.

Generally, a scalpel is a surgical instrument for cutting through skin and/or other soft tissues during surgeries. U.S. Pat. No. 2,650,426 to Montelius, U.S. Pat. No. 5,055,106 to Lundgren and U.S. Pat. No. 5,078,724 to Takase describe examples of scalpels known in the prior art. These surgical scalpels comprise basically a cutting edge, such as a blade attached to a handle. Due to problems such as, but not limited to, the application of too much pressure on the cutting area, trauma, and scaring associated with such surgical scalpels, ultrasonic scalpels were developed. Ultrasonic scalpels are well known in the art and used widely for surgical operations. An ultrasonic scalpel can reduce the amount of pressure needed during cutting and may eliminate some of the trauma associated with such pressure and with the surgery in general.

Furthermore, using ultrasonic scalpels can reduce the scarring associated with surgical operations compared to ordinary scalpels lacking ultrasound energy. Examples of related devices are disclosed in U.S. Pat. No. 5,167,725 to Clark et al, U.S. Pat. No. 5,263,957 to Davison, U.S. Pat. No. 5,324,299 to Davison et al, and U.S. Pat. No. 6,514,267 to Jewett. Jewitt discloses an ultrasonic scalpel having a blade mounted at a 1.5 to 2 degree angle from the device axis. Additionally, these devices teach the use of dull blades for the ultrasonic scalpel cutting edge stating that duller blades are more effective than standard sharp blades when used with ultrasound energy.

A disposable blade is preferred in surgical use because of its convenience, ease of use and low cost. Prior art blades are usually fabricated from materials such as titanium or aluminum believed to facilitate transmission of ultrasonic energy. Prior art devices require blades that are expensive to manufacture thereby preventing their use as disposable or single-use products.

Furthermore, the prior art ultrasonic devices fail to effectively cut through skin and/or soft tissue without damaging the skin and/or soft tissue. This is because most prior art ultrasonic scalpels are designed only for longitudinal motions which are insufficient for cutting without damaging the skin and/or soft tissue. Such trauma to viable tissue can impede the healing potential of such skin and/or tissue. Necrosis (i.e., death of cells and living tissue) also can result from such damage to the skin and/or soft tissue. Necrosis is caused by the continuous friction created from having the blade's cutting edge and/or surface in continuous contact with the skin and/or tissue leading to acoustic burn created from this friction. Such continuous contact is the outcome of having surgeons using a device that makes repeated longitudinal motions with the blade's cutting edge during surgery. This often results in irreversible damage to the skin and/or tissue.

Furthermore, the use of titanium and/or aluminum materials and the design of the blades in prior art ultrasonic scalpels result in blades that are very expensive to replace. Therefore, there is a need for an ultrasonic scalpel that is compatible with a blade that can be manufactured at a sufficiently low cost to be used as a disposable or single-use blade.

### SUMMARY OF THE INVENTION

The present invention is directed towards an ultrasound device for performing surgical operations such as, but not limited to cutting through skin and/or other soft tissues. The device applies ultrasound energy to a cutting edge, such as a surgical blade thereby causing bending motions and longitudinal motions that induce better cutting efficiency and less necrosis formation. The device in accordance with the present invention may meet the above-mentioned needs and also provide additional advantages and improvements that will be recognized by those skilled in the art upon review of the present disclosure.

The device of the present invention comprises an ultrasound generator, a housing, an ultrasound transducer, a transducer horn at the distal end of the ultrasound transducer, a retaining member, and a blade located off-center to the axis of the transducer horn. Ultrasonic energy transmitted through the transducer horn to the blade causes ultrasonic vibrations at the blade. The ultrasonic vibrations at the blade cause the blade to move longitudinally and also laterally, creating a bending wave motion to the blade. The longitudinal waves are vibrations propelling the blade to move forward and backward. Bending waves, created by the placement of the blade off-center to the axis of the transducer horn, aids cutting and separation of skin and/or other soft tissues from the blade thus preventing the side surface of the blade from having continuous contact with skin and/or soft tissue, hence the blade surface is not in continuous contact with the tissue and/or skin. The bending waves decrease and/or eliminate the necrosis of the tissue because the blade surface is not in continuous contact with the skin and/or soft tissue.

The blade of the device of the present invention is preferably made with stainless steel, although the blade may be fabricated from other metals such as, but not limited to aluminum and/or titanium, ceramic, plastic and/or other materials that may be suitable for the purpose. The surgical blade is securely fastened to the slot located at the distal end of the transducer horn. Affixing the blade to the slot effectively transmits ultrasound energy to the blade. The blade may come in different shapes and sizes depending on the surgical procedure to be performed. Typical configurations of the scalpel blade edge profile include linear, curvilinear, serrated either alone or in various combinations.

Because of the risks and costs associated with materials and supplies used in surgery, disposable and single use materials and supplies are used whenever practical. While the present invention in its preferred embodiment includes a stainless steel blade, prior art ultrasonic scalpel devices teach away from the use of stainless steel; an example is U.S. Pat. No. 5,167,725 to Clark et al. These prior art devices teach that the blade element be formed from a material selected from aluminum and/or titanium. Although the device of the present invention may use aluminum and/or titanium, the preferred embodiment of the blade of the present invention is stainless steel. Advantages of utilizing stainless steel for the blade of the present invention include cost and strength. Stainless steel is considerably cheaper to use and decreases the cost associated with purchasing the device when compared to titanium. Therefore the blade can be disposed after single-use, making the present invention more convenient and less expensive to use than prior art devices.

In addition, titanium is brittle in nature and tends to crack relatively easily when used for ultrasound applications. Stainless steel is significantly less brittle than titanium when ultrasonic energy is applied to it greatly simplifying blade fabrication.

Furthermore, prior art ultrasound scalpel devices teach away from using sharp blades, by stating that the application of ultrasonic power to dull blades enhances the sharpness of the blade and thus is preferable; an example is U.S. Pat. No. 5,263,957 to Davison. The present invention utilizes sharp surgical blades coupled with ultrasonic energy for cutting through skin and/or soft tissues.

One of the major advantages of the present invention is the improved and effective cutting of skin and/or soft tissue. Generally, cutting through skin with an ultrasonic scalpel utilizing only longitudinal waves may cause skin and/or soft tissue to come in continuous contact with ultrasonic energy which may lead to necrotic tissues. However, the introduction of bending waves to the surgical blade with the placement of the blade off-center of the axis of the transducer horn reduces and/or eliminates the blade surface from being in continuous contact with skin and/or tissue.

One aspect of the device of the present invention may be to provide an ultrasonic scalpel that is suitable for use with stainless steel blades.

One aspect of the device of the present invention may be to provide an ultrasonic scalpel that is suitable for use with surgical blades of a design that is inexpensive to produce.

One aspect of the device of the present invention may be to provide an ultrasonic scalpel that may effectively cut through skin and/or soft tissue with ease and/or less trauma using disposable blades.

Another aspect of the device of the present invention may be to prevent, reduce and/or eliminate necrosis of tissues.

Another aspect of the device of the present invention may be to create and/or transfer bending waves to the blade which physically flex the blade.

Another aspect of the device may vary the characteristic bending of the blade by varying the diameter of segments of the horn.

These and other aspects of the invention will become more apparent from the written description and figures below.

### BRIEF DESCRIPTION OF THE INVENTION

The present invention will be shown and described with reference to the drawings of preferred embodiments and will be clearly understood in details.

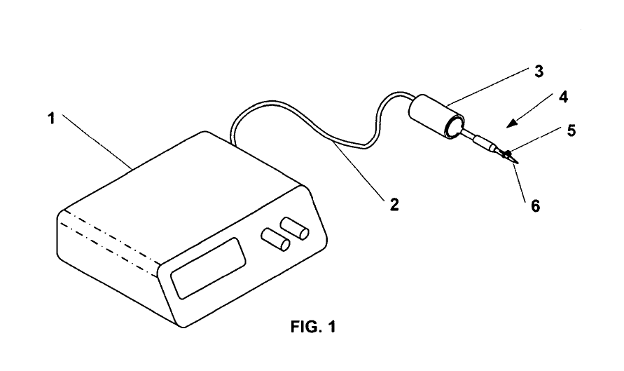

FIG. 1 depicts the entire ultrasonic scalpel device of the present invention.

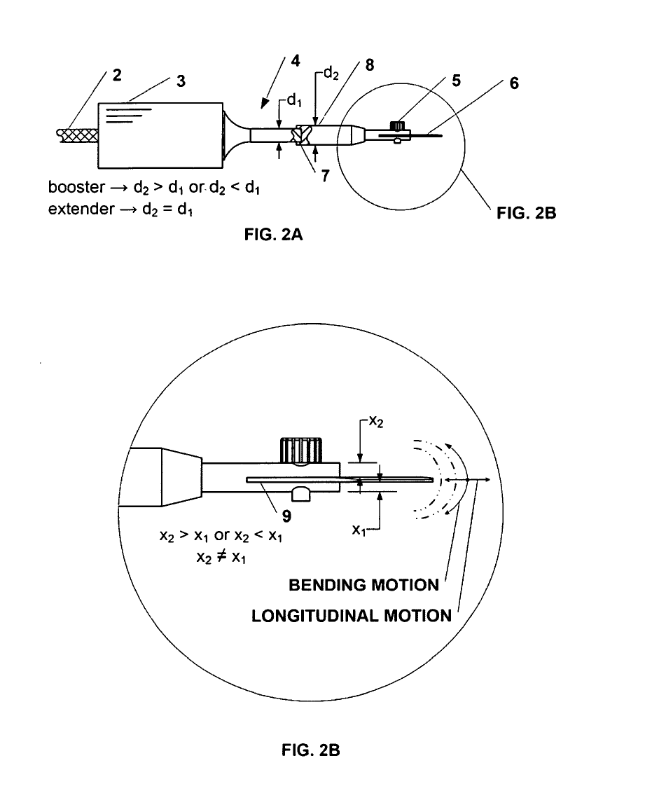

FIG. 2A depicts a cross-sectional view of the device of the present invention.

FIG. 2B depicts a cross-sectional view the distal end of the device of the present invention.

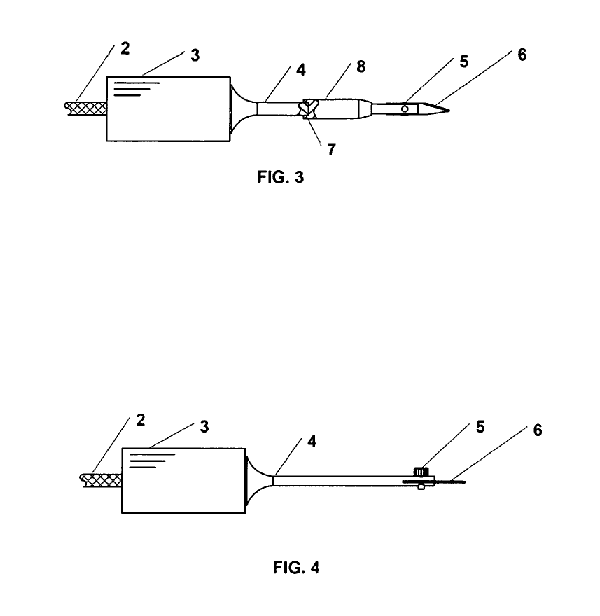

FIG. 3 depicts a side view depicting the ultrasonic scalpel of FIG. 2A.

FIG. 4 depicts a cross-sectional view of an alternative embodiment of the ultrasonic scalpel depicted in FIG. 2A.

### DETAILED DESCRIPTION OF THE INVENTION

The present invention is an ultrasonic scalpel that may be used for several surgical operations including cutting through skin and/or soft tissues. Preferred embodiments of the present invention are illustrated in the figures and described in detail below.

FIG. 1 depicts the entire ultrasonic scalpel device of the present invention. The device of the present invention comprises an ultrasound generator 1, a power cord 2 connected to ultrasound generator 1, a housing 3, an ultrasound transducer within housing 3, an ultrasound transducer horn 4 at the distal end of the ultrasound transducer, a retaining member 5, and a surgical blade 6. The ultrasound transducer within a housing 3 may be mechanically connected to the ultrasound horn 4 by threading, welding or other means as would be understood by those skilled in the art upon review of this disclosure.

The preferred embodiment comprises an ultrasound transducer imbedded within a housing 3 that is directly connected to the ultrasound horn 4 to comprise a single piece without mechanical interface; alternative embodiments may comprise an ultrasound transducer that is connected to the ultrasound horn 4 by a mechanical interface. The housing 3, located at the distal end of the power cord 2, serves as the handle of the ultrasonic scalpel. Surgeons and/or users of the ultrasonic scalpel may hold the housing 3 during surgical operations. The housing 3 provides a surface appropriate for hand manipulation by the surgeon and/or user while preventing direct contact with vibrations from the ultrasound energy. Affixed to distal end of ultrasound horn 4 is a surgical blade 6. The proximal end of blade 6 is located off-center the central axis of ultrasound horn 4 by retaining member 5. The retaining member 5 for releasably securing the surgical blade 6 may include a threaded screw, bolt, captive bolt, compressed spring, leaf spring, bayonet or snap fastener or other attachment means well known in the art.

The ultrasound waves emitted from the ultrasound transducer employed in the present invention may vary with respect to frequency; approximately 15 kHz to 20 MHz. The ultrasound waves employed may also vary with respect to amplitude; approximately between 1 micron and 300 microns.

FIG. 2A depicts an expanded cross-sectional view of the device of the present invention. As the ultrasound transducer within a housing 3 is activated, ultrasound energy is transmitted through transducer horn 4 to surgical blade 6. The proximal end of the transducer horn 4 preferably has a variable diameter segment that necks down to a constant diameter segment with a diameter d₁. The constant diameter segment is located adjacent the necked down variable diameter segment. The constant diameter segment may then be maintained through to the distal end of the transducer horn 4. Alternatively a portion of the transducer horn 4 may be configured as a booster portion 8 having a diameter d₂ that is not equal to diameter d₁. Accordingly, when present, diameter d₂ of booster portion 8 may be greater than or less than diameter d₁.

Ultrasonic energy may be delivered through optional booster portion 8 to the distal end of transducer horn 4 to surgical blade 6. Transducer horn 4 may be configured as a single piece with surgical blade 6. Booster portion 8 may be a single piece integral to the transducer horn 4 without a mechanical interface. Alternatively, the booster portion 8 may be detachable from the transducer horn 4. The preferred embodiment comprises booster portion 8 which may act as a booster portion of the transducer horn 4 by attachment means 7. The attachment means 7 attaching the transducer horn 4 to booster portion 8 may be integrally fixed or may be such as to allow the booster portion 8 to be removed and replaced by the surgeon and/or user. Alternative attachment means 7 for booster portion 8 and/or the surgical blade 6 may include a threaded axial screw or bolt portion, compressed spring, leaf spring, bayonet or snap fastener or other attachment means well known in the art. The ultrasonic transducer, booster portion 8 and/or the surgical blade 6 may be disengaged from the transducer horn 4 so that the appropriate units may be separately sterilized after each use. The transducer horn 4 and booster portion 8 may be fabricated from metals such as, but not limited to titanium, aluminum and/or steel.

An insertable extender portion having d₂=d₁ makes it possible for surgeons and/or users to extend the length of the transducer horn without changing the ultrasound wave characteristics to reach narrow and/or closed spaces during surgery. Booster portion 8 may change the displacement of the transducer horn 4 by increasing the displacement of the transducer horn 4. Booster portion 8 may be used when the diameter of the distal end of the transducer horn 4, represented as diameter d₁, is greater than the diameter of booster portion 8, represented as diameter d₂ (d₂>d₁). Alternatively, booster portion 8 may act as a booster where the diameter of the distal end of the transducer horn 4 is less than the diameter of booster portion 8 (d₂<d₁). Changing the ratio of the diameters will affect the lateral and transverse movement of the surgical blade without changing the characteristics of the ultrasound radiation providing increased operational flexibility of the device.

FIG. 2B depicts a cross-sectional view of the distal end of tie device of the present invention. The distal end of the transducer horn 4 is designed to accommodate a surgical blade 6 which is affixed to it by a retaining member 5. Retaining member 5 secures surgical blade 6 to distal end of transducer horn 4. The distal end of transducer horn 4 may comprise an opening such that the opening may act as a slot 9 wherein surgical blade 6 may be inserted and securely affixed by retaining member 5. Retaining member 5 secures surgical blade 6 into slot 9. Slot 9 at the distal end of transducer horn 4 may be placed parallel to and off-center from the axis of transducer horn 4. The proximal end of surgical blade 6 is also placed off-center the axis of transducer horn 4 into slot 9. Viewing along the axis of the transducer horn 4 from its proximal end defines a right side and left side for the transducer horn and booster portion of the transducer horn. The distance between the slot 9 and the component right side is shown in FIG. 2B as distance x₁. The distance between the slot 9 and the component left side is shown in FIG. 2B as distance x₂. With the present invention, slot 9 is parallel to the transducer horn central axis, but slot 9 is not on the transducer horn central axis and distance x₁ is not equal to distance x₂. Creation of bending waves is made possible because of the placement of surgical blade 6 off-center the axis of transducer horn 4. Activating the ultrasound transducer transmits ultrasound energy from the transducer horn 4 to surgical blade 6. Ultrasound energy transmitted to surgical blade 6 causes bending waves to be created due to the placement of surgical blade 6 off-center the axis of transducer horn 4. The distance between slot 9 and the side walls at the distal end of transducer horn 4 may not be equal.

Surgical blade 6 is also able to exhibit longitudinal waves along with bending waves, as shown by the directional arrows during use by the surgeon and/or user.

Removal of surgical blade 6 for disposing after surgery may be accomplished by the surgeon and/or user turning retaining member 5 head with his/her fingers as if he/she were opening the top of a water bottle. Retaining member 5eases the disposal of used surgical blades by simply turning the retaining member 5 head and releasing surgical blade hence preventing surgeon and/or user from coming in contact with surgical blade during disposal. Although retaining member 5 is a preferred embodiment for the device of the present invention, other means of attachment may be used as would be understood by those skilled in the art upon review of this disclosure.

The transverse motion of the distal end of the surgical blade 6 creates a bending motion of the surgical blade 6. The low frequency bending waves may assist to spread the tissue apart creating a wider incision for a given blade. This motion minimizes tissue contact and prevents excessive injury of the tissue as well as minimizing heating of the preferred embodiment of the material of the surgical blade. This allows stainless steel to be used as a material of fabrication for the blade. This bending motion also improves cutting action somewhat similar to the improvement noted on a serrated blade compared to a straight edged blade.

FIG. 3 depicts a side view depicting the ultrasonic scalpel of FIG. 2A. Surgical blade 6 may be fabricated from materials such as, but not limited to aluminum and/or titanium, steel, ceramic, plastic and/or other materials that may be suitable for the purpose. However, the preferred embodiment comprises surgical blade 6 made from stainless steel alloys. Alloys of aluminum and titanium also provide known advantages for use in ultrasonic surgical blades. Surgical blade 6 may be of different shapes and sizes including straight, rounded, curved, serrated or other combinations, depending on the type of surgery being performed on the patient. Surgical blade 6 may be pre-fitted to transducer horn 4 and may be removed, discarded and replaced as needed. The cutting edge may be placed along a portion of one edge as shown, or may be placed on multiple edges or sides. The cutting edge of surgical blade 6 may be preferably sharp although it is known in the art that dull blades may also be used. A surgical blade 6 with a combination sharp portion and dull portion may also be used.

FIG. 4 depicts a cross-sectional view of an alternative embodiment of the ultrasonic scalpel depicted in FIG. 2A shown with booster portion 8. Surgical blade 6 may be connected directly to transducer horn 4 as a single piece with no mechanical interface and/or component acting as extender, booster and/or combination thereof. As the ultrasound transducer within a housing 3 is activated, ultrasound energy is transmitted through transducer horn 4 to a surgical blade 6. Transducer horn 4 may deliver ultrasonic energy directly to the surgical blade 6. Surgical blade 6 may be located at the distal end of transducer horn 4.

Although specific embodiments have been illustrated and described herein, it will be appreciated by those of ordinary skill in the art that any arrangement that is calculated to achieve the same purpose may be substituted for the specific embodiments shown. It is to be understood that the above description is intended to be illustrative and not restrictive. Methods are not intended to be restricted to the order described. Combinations of the above embodiments and other embodiments will be apparent to those having skill in the art upon review of the present disclosure. The scope of the present invention should be determined with reference to the appended claims, along with the full scope of equivalents to which such claims are entitled.

I claim:

1. An ultrasonic scalpel comprising:
a. an ultrasound generator;
b. an ultrasound transducer producing ultrasonic energy within a housing;
c. the ultrasound transducer in communication with the ultrasound generator;
d. a transducer horn connected to the ultrasound transducer, the transducer horn having a center axis; and
e. a surgical blade connected to the transducer horn the surgical blade being located parallel to and offset from the transducer horn center axis.

2. The ultrasonic scalpel of claim 1 further comprising a retaining member for releasably attaching the surgical blade to the transducer horn.

3. The ultrasonic scalpel of claim 1 further characterized by the ultrasound transducer as an extender to said transducer horn.

4. The ultrasonic scalpel of claim 1 further characterized by the surgical blade being disposable.

5. The ultrasonic scalpel of claim 1 further characterized by the surgical blade being constructed of a material selected from the group consisting of stainless steel, titanium alloy and aluminum alloy.

6. The ultrasonic scalpel of claim 1 further characterized by the surgical blade producing a lateral bending motion.

7. An ultrasonic scalpel comprising:
a. an ultrasound generator;
b. an ultrasound transducer producing ultrasonic energy within a housing;
c. the ultrasound transducer in communication with the ultrasound generator;
d. a transducer horn connected to the ultrasound transducer, the transducer horn having a center axis and a constant diameter segment; and
e. a surgical blade connected to the transducer horn the surgical blade being located parallel to and offset from the transducer horn center axis.

8. The ultrasonic scalpel of claim 7 wherein the transducer horn contains a booster portion having a diameter not equal to the transducer horn constant diameter segment.

9. The ultrasonic scalpel of claim 8 wherein the transducer horn contains a booster portion diameter less than the transducer horn constant diameter segment.

10. The ultrasonic scalpel of claim 8 wherein the transducer horn contains a booster portion diameter greater than the transducer horn constant diameter segment.

11. The ultrasonic scalpel of claim 8 further comprising a retaining member for releasably attaching the surgical blade to the transducer horn.

12. The ultrasonic scalpel of claim 8 further characterized by the surgical blade being disposable.

13. The ultrasonic scalpel of claim 8 further characterized by the surgical blade being stainless steel.

14. The ultrasonic scalpel of claim 8 further characterized by the surgical blade having a lateral bending motion.

15. The ultrasonic scalpel of claim 8 wherein the booster portion is interchangeable.

16. The ultrasonic scalpel of claim 8 further characterized by the surgical blade having a sharp edge.
